(19) United States
(12) Patent Application Publication
(10) Pub. No.: US 2009/0234150 A1
(43) Pub. Date: Sep. 17, 2009

(54) Use of ppar-alpha agonists to treat skeletal muscle wasting disorders

(76) Inventor: Peter Carmeliet, Blanden (BE)

Correspondence Address: NIXON & VANDERHYE, PC, 901 NORTH GLEBE ROAD, 11TH FLOOR, ARLINGTON, VA, 22203, US

(21) Appl. No.: 12/309,321

(22) PCT Filed: Jul. 10, 2007
(86) PCT No.: PCT/EP2007/057014

§ 371 Date: Apr. 7, 2009

(30) Foreign Application Priority Data
Jul. 14, 2006 (EP) 06117242.5

(51) Int. Cl. C07C 69/738

(52) U.S. Cl. 560/52

(57) ABSTRACT

The present invention relates to the field of muscle pathologies, more particularly to the field of diseases where skeletal muscle damage and muscle loss occurs. The invention shows that peroxisome proliferator-activated receptor (PPAR)-alpha agonists, such as fibrates, can be used to prevent skeletal muscle degeneration, more particularly ischemic skeletal muscle degeneration.

### FIELD OF THE INVENTION

The present invention relates to the field of muscle pathologies, more particularly to the field of diseases where skeletal muscle damage and muscle loss occurs. The invention shows that peroxisome proliferator-activated receptor (PPAR)-alpha agonists, such as fibrates, can be used to prevent skeletal muscle degeneration, more particularly ischemic skeletal muscle degeneration.

### BACKGROUND OF THE INVENTION

Peroxisome proliferator-activated receptors (PPARs), originally identified in Xenopus as receptors that induce the proliferation of peroxisomes in cells, are members of the nuclear hormone receptor family of transcription factors that mediate a variety of cellular processes, including glucose and lipid metabolism, inflammatory responses, and regulation of apoptotic cell death. They act by binding to specific peroxisome proliferator-response elements (PPREs) on target genes. Three forms of PPARs have been described, which are designated as alpha, delta, and gamma forms. They contain a DNA binding domain and a ligand-binding domain. The DNA-binding domain contains two zinc finger patterns, which binds to the regulator region of DNA when the receptor is activated. The ligand-binding domain has an extensive secondary structure of several alpha-helices and a beta-sheet. Each form is expressed in different tissues and can be activated by different ligands, most of them being specific for one form of PPAR. PPAR-alpha is expressed in skeletal muscle, liver, kidney, and endothelial cells and regulates lipoprotein metabolism. Its transcriptional activity is enhanced in the presence of insulin. PPAR-delta is shown to be widely distributed in animal tissues and is reported to be involved in oligodendrocyte differentiation. It is expressed to higher levels in brain, adipose tissue, and skin. PPAR-gamma is the most studied isoform and plays a critical role in adipocyte differentiation and fat deposition. All PPARs dimerize with the retinoid-X-receptor (RXR) and bind to specific regions on the DNA of target genes. These DNA sequences are termed PPREs (peroxisome proliferator response elements). Generally, this sequence occurs in the promotor region of a gene, and when the PPAR binds its ligand, transcription of targets genes is increased or decreased, depending on the gene. The RXR also forms a heterodimer with a number of other receptors: the vitamin D receptor and the thyroid hormone receptor. When induced by a natural ligand a conformational change occurs in the heterodimer and co-repressor complexes are displaced. This promotes binding of the PPAR-RXR complex to specific DNA sequences, PPRE, located in the regulatory regions of target genes. PPREs are commonly found in genes involved in lipid metabolism and energy balance, including those encoding lipoprotein lipase, adipocyte fatty acid binding protein, fatty acyl-CoA synthase, glucokinase, and glucose transporter GLUT4. The function of PPARs is modified by the exact shape of their ligand-binding domain and by a number of co-activators and co-repressors, the presence of which can stimulate or inhibit receptor function. The natural ligands for the PPARs are free fatty acids and eicosanoids. PPARγ is activated by PGJ₂ (a prostaglandin). In contrast, PPARα is activated by leukotriene B₄.

PPAR-alpha agonists such as fibrates (examples of fibrates are gemfibrozil, fenofibrate, bezafibrate and ciprofibrate) are beneficial in the prevention of ischemic heart disease because they lower plasma triglycerides and cholesterol levels in individuals. In addition, PPAR-alpha agonists such as fenofibrate protect the heart (Yue T L et al (2003) Circulation 108(19):2393-9) and kidney (Portilla D et al (2000) Am J Physiol Renal Physiol 278(4): F667-75) from ischemia/reperfusion injury and also protect against cerebral injury (Delplanque D et al (2003) J. Neurosci 23(15): 6264-71). In the present invention we have surprisingly found that PPAR-alpha agonists also prevent skeletal muscle wasting in a murine model for acute limb ischemia. We show that PPAR-alpha agonists can be used to manufacture a medicament to prevent skeletal muscle loss in muscle wasting disorders.

### AIMS AND DETAILED DESCRIPTION OF THE INVENTION

Metabolic and functional impairments in skeletal muscle frequently occur. These range from the symptoms of pain and fatigue to pathological disorders that may result in muscle wasting. Notwithstanding these symptoms and features of skeletal muscle disease occur in diverse conditions, each with different aetiologies, one common denominator in skeletal muscle diseases is the loss of functional skeletal muscle. In the present invention we have shown striking skeletal muscle protection after acute hind limb ischemia provided the hind limb was pretreated with a PPAR-alpha agonist. Pretreated mice that received a fibrate display a significant preservation of myofiber viability in acute hindlimb ischemia, a condition normally leading to severe skeletal muscle necrosis. Thus the present invention relates to the use of PPAR-alpha agonists for the treatment of disorders involving diseases where skeletal muscle loss occurs, more particularly to the treatment of diseases where skeletal muscle loss occurs due to ischemia.

The wording ‘diseases where skeletal muscle loss occurs’ refers to skeletal muscle cells that have been exposed for example to an ischemic insult, or for example skeletal muscle cells that possess a reduced glycolytic rate, or for example skeletal muscle cells that have been exposed to serum deprivation or for example skeletal muscle cells that lack enervation or for example skeletal muscle cells that have been immobilized for a long time. The wording ‘diseases where skeletal muscle loss occurs’ also refers to muscle disorders that directly affect skeletal muscles. This includes the muscular dystrophies, the structural myopathies, the inflammatory myopathies, myotonic disorders, channelopathies and metabolic muscle diseases. The muscular dystrophies are among the most recognizable forms of neuromuscular disorders. Duchenne, Becker, and Emery-Dreifuss are among the most common forms. Also included in this category are the various forms of Limb-Girdle muscle dystrophies. The structural myopathies make up a relatively rare group of muscle disorders including central core myopathies, nemaline myopathies, and other forms of muscle disease. The acquired inflammatory myopathies make up a group of disorders that are not specifically related to a genetic mutation. Multiple genes may affect the progression of such disorders, but generally forms of muscle weakness including dermatomyositis, inclusion body myositis, and polymyositis are considered to be caused by other factors. Myotonic disorders make up a very rare group of muscle diseases, including rippling muscle disease and Brody disease. This group does not contain the more common myotonic dystrophies. The skeletal muscle channelopathies are a group of genetic disorders affecting the ion channels of muscle membranes, which includes such muscle diseases as myotonia congenita, Andersen syndrome, and paramyotonia congenita. The metabolic diseases of muscle are a group of neuromuscular disorders that involve the muscle's cellular machinery which processes the energy required for those muscles to function. This group includes disorders such as the mitochondrial myopathies, Pompe's disease, and other forms of glycogenoses. Loss of skeletal muscle or degeneration of skeletal muscle is also a common condition of elderly people which is designated as sarcopenia.

‘Degeneration of skeletal muscle cells’ is herein equivalent to the terms ‘necrotic skeletal muscle cell death’, ‘apoptotic muscle cell death, ‘skeletal muscle cell atrophy’, ‘skeletal fiber injury’, ‘skeletal muscle wasting’ and also more generally as ‘skeletal muscle loss’.

The present invention provides in one embodiment the use of an effective amount of a peroxisome proliferator-activated receptor (PPAR)-alpha agonist for the manufacture of a medicament to treat and/or to prevent skeletal muscle loss. The wording “to treat and/or to prevent skeletal muscle loss” can be interchanged with the wording “to treat and/or to prevent skeletal muscle degenerative diseases” or with the wording “to treat and/or to prevent skeletal muscle damage” or with the wording “to treat and/or to prevent muscle wasting disorders (diseases)”. In the present invention a PPAR-alpha agonist can be used in combination with a PPAR-delta or in combination with a PPAR-gamma agonist. Indeed, several agonists are known with a dual specificity for PPAR-alpha and PPAR-gamma which can thus also be used in the present invention.

In yet another embodiment an effective amount of a fibrate is used for the manufacture of a medicament to treat and/or to prevent skeletal muscle loss.

In yet another embodiment an effective amount of a fenobrate is used for the manufacture of a medicament to treat and/or to prevent diseases where skeletal muscle loss occurs.

Several PPAR-alpha agonists are described in the art which can be used in the context of the present invention. Non-limiting examples are aliphatic compounds described in WO03004484 (Maruha corporation), heterocyclic compounds described in WO03043985 (Novartis AG), arylthiazolidinedione and aryloxazolidinedione derivatives described in WO00078312 and WO00078313 (Merck & Co, Inc), bicyclic compounds described in WO05095363 (Daiichi Pharmaceutical corporation), phenoxyacetic acid derivative described in WO05095364 (Daiichi Pharmaceutical corporation), aryloxyacetic acids described in WO01060807 and U.S. Pat. No. 6,569,879 (Merck & Co, Inc), 2-aryloxy-2-arylalkanoic acids as described in WO02064094 (Merck & Co, Inc), aniline derivatives as described in WO04111020 (F. Hoffmann-la Roche AG), benzopyrancarboxylic acid derivatives as described in U.S. Pat. No. 6,645,997 and U.S. Pat. No. 6,713,508 (Merck & co, Inc), thiazole-2-carboxamide derivatives as described in WO05037804 (Smithkline Beecham Corporation), oleoylethanolamide-like compounds as described in WO05002524 (The Regents of the University of California), heteroaryl derivatives as described in WO05049606 (F. Hoffmann-la Roche AG), pyrazolyl indolyl derivatives as described in WO05085235 (F. Hoffmann-la Roche AG), indolyl derivatives substituted with a thiazole ring as described in WO05005423 (F. Hoffmann-la Roche AG), benzannelated compounds as described in WO05049572 (F. Hoffmann-la Roche AG), pyrazole phenyl derivatives as described in WO05105754 (F. Hoffmann-la Roche AG), substituted heteroaryl- and phenylsulfamoyl compounds as described in US2005288340 (Pfizer Inc), dithiolane derivatives as described in WO01025226 (Bethesda Pharmaceuticals Inc), analogues of resveratrol such as pterostilbene as described in US200657231, phenyl derivatives as described in WO05049573 (F. Hoffmann-la Roche AG), substituted phenylpropionic acid derivatives as described in U.S. Pat. No. 6,506,797 and U.S. Pat. No. 6,949,259 (Kyorin Pharmaceutical Co, Ltd), tetrahydroisoquinoline derivatives as described in U.S. Pat. No. 6,987,118 (Pfizer Inc).

Several patents claim distinct formulations of fibrates. Non-limiting examples are nanoparticulate fibrate formulations as described in US2005276974 (Elan Pharma International Ltd), stabilised fibrate microparticles as described in WO02024193 (RTP Pharma Inc), self-emulsifying formulations of fenofibrate and derivatives with improved bioavailability as claimed in U.S. Pat. No. 7,022,337 (Shire Laboratories Inc), a granular medicine based on fenofibrate as claimed in U.S. Pat. No. 480,007, a controlled release form of fenofibrate as claimed in U.S. Pat. No. 4,961,890 (Ethypharm), a novel dosage form of fenofibrate as claimed in U.S. Pat. No. 4,895,726 (Fournier Innovation et Synergie, Fr), a novel fenofibrate galenic formulation as claimed in EP1112064B1 (CLL Pharma), fenofibrate pharmaceutical compositions having high bioavailability as claimed in U.S. Pat. No. 6,589,552, U.S. Pat. No. 6,596,317, U.S. Pat. No. 6,074,670, U.S. Pat. No. 7,037,529 and U.S. Pat. No. 7,041,319 (Laboratoires Foumier, Fr), fenofibrate tablets as described in US2004115264, oral dosage forms comprising fenofibrate as described in WO03092659 (Skyepharma Canada Inc). All these fibrate compositions can be used in the present invention for the manufacture of a medicament to prevent and/or to treat diseases where skeletal muscle loss occurs.

A serious indication where skeletal muscle degeneration takes place is due to ischemic insults. For example it has become increasingly recognized that skeletal muscle atrophy is common in patients with chronic pulmonary disease (COPD). Another example where skeletal muscle atrophy occurs is critical limb ischemia (CLI) which is a disease manifested by sharply diminished blood flow to the legs. Up to 10 million people in the US alone suffer from severe leg pain (claudication) and non-healing-ulcers (peripheral vascular disease), both of which can ultimately lead to CLI. The most common causes that can lead to CLI are atherosclerosis and embolization (e.g. a clot that has been ejected from a failing heart, or from an aneurysm in the aorta, into the leg). The present invention also shows that PAR-alpha agonists can be used for skeletal muscle preservation during transient ischemic conditions which can occur for example during an operation. Yet another class of skeletal muscle degenerative diseases are muscle pathologies associated with a reduced glycolytic rate such as McArdle's disease and phosphofructokinase disease (PFKD). Yet another class comprises muscle atrophy which occurs due to muscle denervation. In such denervation atrophy there occurs a lack of tonic stimuli and muscle cells become atrophic. Causes of denervation atrophy include localized loss of nerve function (neuritis) or generalized loss of the entire motor unit. After denervation, muscles become rapidly atrophic and 50% of muscle mass could be lost in just a few weeks. Another class of such diseases comprises muscle degeneration which occurs due to immobilization. ‘Immobilization’ means here that the skeletal muscle system is unloaded because of for example prolonged space flight, during conservative treatment after sports injuries or by a plaster cast after orthopedic surgery. This immobilization causes a serious atrophy of muscle mass leading to a decrease in physical performance and high power output capacity. Yet another class of such diseases where muscle degeneration takes place comprises muscular dystrophies. These disorders include a progressive wasting of skeletal muscle. The most common examples are Duchenne and Becker muscular dystrophy. Yet another class of conditions were muscle degeneration takes place comprises critical illness. Critical illness (e.g. burns, sepsis) is associated with a serious muscle wasting and muscle weakness.

The present invention not only aims at using a PPAR-alpha agonist for the manufacture of a medicine to treat humans but also aims at using these compositions for veterinary diseases and conditions. Common causes of myopathies (degenerative diseases of muscle) in animals which can also be treated with a PPAR-alpha agonist are: (1) metabolic myopathies (e.g. porcine stress syndrome, malignant hyperthermia and pale soft exudative pork), (2) exertional myopathies which comprise a group of diseases which result in severe muscle degeneration following strenuous exercise (e.g. azoturia and tying-up in horses, greyhound myopathy in dogs, capture myopathy in wild animals and compartment syndrome in poultry), (3) traumatic myopathies (e.g. Downer syndrome which is an ischemic necrosis of ventral and limb muscles following prolonged recumbency (disease/anesthesia) and Crush syndrome).

The term ‘medicament to treat’ relates to a composition comprising PPAR-alpha agonists as described above and a pharmaceutically acceptable carrier or excipient (both terms can be used interchangeably) to treat skeletal muscle degenerative diseases. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. The ‘medicament’ may be administered by any suitable method within the knowledge of the skilled man. One route of administration is parenterally. In parental administration, the medicament of this invention will be formulated in a unit dosage injectable form such as a solution, suspension or emulsion, in association with the pharmaceutically acceptable excipients as defined above. However, the dosage and mode of administration will depend on the individual. Another preferred route of administration is oral administration. Generally, the medicament is administered so that the PPAR-alpha agonist of the present invention is given at a dose between 1 pg/kg and 1 g/kg, more preferably between 100 μg/kg and 0.5 g/kg. It can be given as a bolus dose. Continuous infusion may also be used and includes continuous subcutaneous delivery via an osmotic minipump. It is clear to the person skilled in the art that the use of a therapeutic composition comprising a PPAR-alpha agonist for the manufacture of a medicament to treat skeletal muscle degenerative diseases can be administered by any suitable means, including but not limited to, parenteral, subcutaneous, intraperitoneal, intrapulmonary, oral and intranasal administration. Parenteral infusions include intramuscular, intravenous, intra-arterial, intraperitoneal, or subcutaneous administration.

The following examples more fully illustrate preferred features of the invention, but are not intended to limit the invention in any way. All of the starting materials and reagents disclosed below are known to those skilled in the art, and are available commercially or can be prepared using well-known techniques.

### EXAMPLE

### 1. Treatment With a PPAR-alpha Agonist Protects Skeletal Muscle Against Ischemic Necrosis

To assess the muscle protective effect of the PPAR-alpha agonist fenofibrate, wild type mice received fenofibrate (0.5 g/kg/day; Cayman Chemical, Ann Arbor, Mich., USA) by gavage, or vehicle alone for 10 days prior to femoral artery ligation. Two days after femoral artery ligation muscle necrosis was subsequently analyzed on histological sections of the crural muscles. We showed that pretreatment of WT mice with fenofibrate prevented ischemic muscle necrosis by 60% (% necrotic area/total cross-sectional area: 86.3±8.2% in vehicle-treated mice versus 34.3±7% in fenofibrate-treated mice; n=4; P=0.002). In accordance with previous reports (Tabernero A et al (2002) BMC Pharmacol 2, 10 and Schoonjans K et al (1996) EMBO J 15, 5336), the fenofibrate dosage used for this experiment did not induce toxic side effects, or weight reduction.

### 2. Treatment of a Murine Model for Human Motoneuron Disease With PPAR-alpha Agonists

The progressive motor neuronopathy (pmn) mutant mouse is an accepted animal model in the art for human motoneuron disease (Schmalbruch H et al (1991) J Neuropathol Exp Neurol 50(3):192-204. Mice that are homozygous for the pmn gene defect appear healthy at birth but develop progressive motoneuron disease, resulting in severe skeletal muscle wasting and respiratory failure by postnatal week 3. The pmn mutant mice are being treated with fenofibrate starting at birth and the muscle loss is monitored.

### 3. Treatment of a Murine Model for Skeletal Muscle Dystrophy With Recombinant Cripto

A mutant mouse model with an X-chromosome-linked muscular dystrophy (mdx) is a reliable animal model that mimics the human Duchenne muscular dystrophy (Tanabe Y et al (1986) Acta Neuropathol (Berl) 69(1-2):91-5) mainly because of the similar histological features. This is a strain of mice arising from a spontaneous mutation (mdx) in inbred C57BL mice. This mutation is X-chromosome-linked and produces viable homozygous animals that lack the muscle protein dystrophin, have high serum levels of muscle enzymes, and possess histological lesions similar to human muscular dystrophy. The mdx mice are being treated with fenofibrate and the muscle wasting is monitored.

### Materials and Methods

### Mouse Model of Limb Ischemia

Limb ischemia was induced by high unilateral right or bilateral ligation of the femoral artery and vein, and of the cutaneous vessels branching from the caudal femoral artery, sparing the femoral nerve. Crural muscles were dissected and processed for histological analysis 2 d or 7 d after ligation.

### Histology

The crural muscles were dissected, fixed in 4% PFA, dehydrated, embedded in paraffin, and sectioned at 10 μm thickness. Microscopic analysis was performed with a Zeiss Axioplan 2 imaging microscope, equipped with an Axiocam HrC camera and KS300 morphometry software (Zeiss). Capillary density, fiber size, and the cross-sectional area of viable and necrotic zones were quantified on eight entire sections (each 320 μm apart) of the crural muscles.

1. Use of a peroxisome proliferator-activated receptor (PPAR)-alpha agonist for the manufacture of a medicament to treat and/or to prevent skeletal muscle diseases where skeletal muscle loss occurs.

2. Use according to claim 1 wherein said skeletal muscle cell loss is due to ischemia, such as occurs in chronic obstructive pulmonary disease and critical limb ischemia.

3. Use according to claim 1 wherein said skeletal muscle cell loss is due to a reduced glycolytic rate, such as occurs in McArdle's disease and PFKD.

4. Use according to claim 1 wherein said skeletal muscle cell loss is due to immobilization, such as occurs in space flights and during a long bed rest.

5. Use according to claim 1 wherein said skeletal muscle cell loss is due to muscle denervation, such as occurs in peripheral neuropathies.

6. Use according to claim 1 wherein said skeletal muscle cell loss is due to a muscle dystrophy, such as occurs in Duchenne muscular dystrophy.

7. Use according to claim 1 wherein said peroxisome proliferator-activated receptor (PPAR)-alpha agonist is a fibrate.

8. Use according to claim 7 wherein said fibrate is fenofibrate.
